SEQUENCE LISTING (18 sequences)

SEQ ID NO: 1 - protein, 108 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 5 aa, Homo sapiens
RYWMS

SEQ ID NO: 4 - protein, 17 aa, Homo sapiens
NIKQDGSEKYYVDSVKG

SEQ ID NO: 5 - protein, 12 aa, Homo sapiens
EGGWFGELAFDY

SEQ ID NO: 6 - protein, 12 aa, Homo sapiens
RASQRVSSSYLA

SEQ ID NO: 7 - protein, 7 aa, Homo sapiens
DASSRAT

SEQ ID NO: 8 - protein, 9 aa, Homo sapiens
QQYGSLPWT

SEQ ID NO: 9 - protein, 139 aa, Artificial Sequence
  Description of Artificial Sequence Synthetic polypeptide
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 10 - protein, 167 aa, Artificial Sequence
  Description of Artificial Sequence Synthetic polypeptide
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 11 - protein, 10 aa, Artificial Sequence
  Description of Artificial Sequence Synthetic peptide
GFTFSSYGMH

SEQ ID NO: 12 - protein, 15 aa, Artificial Sequence
  Description of Artificial Sequence Synthetic peptide
VIWYDGSNKYYADSV

SEQ ID NO: 13 - protein, 16 aa, Artificial Sequence
  Description of Artificial Sequence Synthetic peptide
DPRGATLYYYYYGMDV

SEQ ID NO: 14 - protein, 11 aa, Artificial Sequence
  Description of Artificial Sequence Synthetic peptide
RASQSINSYLD

SEQ ID NO: 15 - protein, 7 aa, Artificial Sequence
  Description of Artificial Sequence Synthetic peptide
AASSLQS

SEQ ID NO: 16 - protein, 9 aa, Artificial Sequence
  Description of Artificial Sequence Synthetic peptide
QQYYSTPFT

SEQ ID NO: 17 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 18 - DNA, 3349 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

(19) United States
(12) Patent Application Publication
(10) Pub. No.: US 2016/0060344 A1
(43) Pub. Date: Mar. 3, 2016

(54) COMBINATION THERAPY FOR PD-L1 NEGATIVE TUMORS

(71) Applicants: Rajesh Narwal, Gaithersburg, MD (US); Marlon C. Rebelatto, Gaithersburg, MD (US); Keith Steele, Gaithersburg, MD (US); Paul Robbins, Gaithersburg, MD (US); Ross Anthony Stewart, Cambridge (GB); John A. Blake-Haskins, Gaithersburg, MD (US); Joyson J. Karakunnel, Gaithersburg, MD (US); Ramy Ibrahim, Gaithersburg, MD (US); Aiman Shalabi, Gaithersburg, MD (US); Alessandra Di Pietro, Gaithersburg, MD (US); Li Shi, Gaithersburg, MD (US); Shengyan Hong, Gaithersburg, MD (US); Paul Stockman, Alderley Park (GB); Marc Ballas, Gaithersburg, MD (US); Mohammed M. Dar, Gaithersburg, MD (US)

(72) Inventors: Rajesh Narwal, Gaithersburg, MD (US); Marlon C. Rebelatto, Gaithersburg, MD (US); Keith Steele, Gaithersburg, MD (US); Paul Robbins, Gaithersburg, MD (US); Ross Anthony Stewart, Cambridge (GB); John A. Blake-Haskins, Gaithersburg, MD (US); Joyson J. Karakunnel, Gaithersburg, MD (US); Ramy Ibrahim, Gaithersburg, MD (US); Aiman Shalabi, Gaithersburg, MD (US); Alessandra Di Pietro, Gaithersburg, MD (US); Li Shi, Gaithersburg, MD (US); Shengyan Hong, Gaithersburg, MD (US); Paul Stockman, Alderley Park (GB); Marc Ballas, Gaithersburg, MD (US); Mohammed M. Dar, Gaithersburg, MD (US)

(21) Appl. No.: 14/838,650

(22) Filed: Aug. 28, 2015

Related U.S. Application Data
(63) Continuation of application No. PCT/EP2015/060523, filed on May 12, 2015.
(60) Provisional application No. 62/043,148, filed on Aug. 28, 2014.
(60) Provisional application No. 62/105,992, filed on Jan. 21, 2015.
(60) Provisional application No. 62/114,336, filed on Feb. 10, 2015.

(51) Int. Cl. C07K 16/28

(52) U.S. Cl. CPC C07K 16/2827; C07K 16/2818; C07K 2317/21; C07K 2317/76; A61K 2039/505

(57) ABSTRACT

The present invention features methods of treating lung cancer (e.g., NSCLC) with an anti-PD-L1 antibody and tremelimumab in a subject identified as having a PD-L1 negative tumor.

### CROSS-REFERENCE TO RELATED APPLICATION

This application claims priority to and the benefit of U.S. Provisional Patent Application Ser. No. 62/043,148, filed Aug. 28, 2014, U.S. Provisional Patent Application Ser. No. 62/105,992, filed Jan. 21, 2015, U.S. Provisional Patent Application Ser. No. 62/114,336, filed Feb. 10, 2015, and International Application No. PCT/EP2015/060523, filed May 12, 2015. The entire contents of each of these applications are hereby incorporated by reference herein.

### SEQUENCE LISTING

The instant application contains a Sequence Listing which has been submitted electronically in ASCII format and is hereby incorporated by reference in its entirety. Said ASCII copy, created on Aug. 24, 2015, is named TRB7-110US1_SL.txt and is 14,077 bytes in size.

### BACKGROUND OF THE INVENTION

Lung cancer is among the most common forms of cancer and is the leading cause of cancer deaths among men and women. More people die of lung cancer annually than of colon, breast, and prostate cancers combined. Non-small cell lung cancer (NSCLC) is the most common form of lung cancer. While the risk of acquiring lung cancer is higher among patients with a history of smoking, lung cancer also affects non-smokers. Improving survival of lung cancer patients remains difficult despite improved medical therapies. Most lung cancer is detected only in advanced stages when therapy options are limited. There is a growing recognition that lung cancer and other malignancies arise from a variety of pathogenic mechanisms. Methods of characterizing these malignancies at a molecular level are useful for stratifying patients, thereby quickly directing them to effective therapies. Improved methods for predicting the responsiveness of subjects having lung cancer, including NSCLC, are urgently required.

### SUMMARY OF THE INVENTION

As described below, the present invention features methods of treating lung cancer (e.g., small cell lung cancer, non-small cell lung cancer) with an anti-PD-L1 antibody and tremelimumab in a subject identified as having a PD-L1 negative tumor.

In particular, the invention generally provides methods for characterizing lung cancer in a subject for PD-L1 expression, thereby quickly directing subjects identified as having PD-L1 negative tumors to anti-PD-L1 antibody and tremelimumab as an effective therapy.

In one aspect, the invention provides a method of treatment that involves administering an anti-PD-L1 antibody and an anti-CTLA4 antibody, or antigen binding fragments thereof, to a patient identified as having lung cancer that is negative for PD-L1. In one embodiment, the lung cancer is small cell lung cancer or non-small cell lung cancer (e.g., squamous cell carcinoma, adenocarcinoma, large cell carcinoma, adenosquamous carcinoma and sarcomatoid carcinoma). In one embodiment, the anti-PD-L1 antibody is MEDI4736. In another embodiment, the anti-CTLA4 antibody is tremelimumab.

In another aspect, the invention provides a method of treatment involving administering MEDI4736 and tremelimumab or antigen binding fragments thereof to a patient identified as having a non-small cell lung cancer that is negative for PD-L1.

In another aspect, the invention provides a method of treatment involving administering between about 1 mg/kg and 20 mg/kg MEDI4736 and between about 1 mg/kg and 10 mg/kg tremelimumab or antigen binding fragments thereof to a patient identified as having lung cancer that is negative for PD-L1.

In another aspect, the invention provides a kit for treating lung cancer (e.g., non-small cell lung cancer), the kit containing tremelimumab, MEDI4736 or antigen binding fragments thereof, and an anti-PD-L1 antibody suitable for use in immunohistochemistry. In one embodiment, the kit further contains immunohistochemistry reagents.

In various embodiments of any of the above aspects or any aspect of the invention delineated herein, the lung cancer is small cell lung cancer or non-small cell lung cancer. In various embodiments of any of the above aspects or any aspect of the invention delineated herein the anti-PD-L1 antibody is MEDI4736. In other embodiments, the anti-CTLA4 antibody is tremelimumab. In various embodiments of any of the above aspects, the non-small cell lung cancer is squamous cell carcinoma, adenocarcinoma, large cell carcinoma, adenosquamous carcinoma or sarcomatoid carcinoma. In various embodiments of any of the above aspects, the treatment is administered every 2 weeks, 3 weeks, or 4 weeks. In various embodiments of any of the above aspects, about 1 mg/kg MEDI4736 and about 1 mg/kg tremelimumab is administered; about 3 mg/kg MEDI4736 and about 1 mg/kg tremelimumab is administered; about 10 mg/kg MEDI4736 and about 1 mg/kg tremelimumab is administered; about 15 mg/kg MEDI4736 and about 1 mg/kg tremelimumab is administered; about 1 mg/kg MEDI4736 and about 3 mg/kg tremelimumab is administered; about 3 mg/kg MEDI4736 and about 3 mg/kg tremelimumab is administered; about 10 mg/kg MEDI4736 and about 3 mg/kg tremelimumab is administered; about 15 mg/kg MEDI4736 and about 3 mg/kg tremelimumab is administered; about 1 mg/kg MEDI4736 and about 10 mg/kg tremelimumab is administered; about 3 mg/kg MEDI4736 and about 10 mg/kg tremelimumab is administered; about 10 mg/kg MEDI4736 and about 10 mg/kg tremelimumab is administered; or about 15 mg/kg MEDI4736 and about 10 mg/kg tremelimumab is administered. In various embodiments of any of the above aspects, the patient is identified as responsive to treatment with an anti-PD-L1 antibody and an anti-CTLA4 antibody, or antigen binding fragments thereof. In various embodiments of any of the above aspects, PD-L1 is detected using immunohistochemistry (e.g., on cancer cells that are formalin fixed and paraffin embedded). In various embodiments of any of the above aspects, the method results in an increase in overall survival (e.g., an increase of weeks, months or years) as compared to the administration of either the MEDI4736 or an antigen-binding fragment thereof or the tremelimumab or an antigen-binding fragment thereof alone. In particular, the increase in survival is more than about 4-6 weeks, 1-2 months, 3-4 months, 5-7 months, 6-8 months, or 9-12 months. In various embodiments of any of the above aspects, the administration of MEDI4736 or an antigen-binding fragment thereof is repeated about every 4 weeks. In various embodiments of any of the above aspects, the administration of tremelimumab or an antigen-binding fragment thereof is repeated about every 4 weeks. In various embodiments of any of the above aspects, the administration of tremelimumab or an antigen-binding fragment thereof is repeated about every 12 weeks. In various embodiments of any of the above aspects, the administration of tremelimumab or an antigen-binding fragment thereof is administered about every 4 weeks for seven administrations and then every 12 weeks. In various embodiments of any of the above aspects, the administration of MEDI4736 or an antigen-binding fragment thereof is by intravenous infusion. In various embodiments of any of the above aspects, the administration of tremelimumab or an antigen-binding fragment thereof is by intravenous infusion. In various embodiments of any of the above aspects, tremelimumab and MEDI4736 are administered concurrently or at different times. In various embodiments of any of the above aspects, tremelimumab and MEDI4736 are administered twenty-four, forty-eight or seventy-two hours apart, 1, 2, or 3 weeks apart, or between 1, 2, and 3 months apart. In various embodiments of any of the above aspects, the non-small cell lung cancer expresses reduced or undectable levels of PD-L1. In various embodiments of any of the above aspects, the non-small cell lung cancer is negative for PD-L1 when less than 25% of cancer cells show PD-L1 staining.

Other features and advantages of the invention will be apparent from the detailed description, and from the claims.

### BRIEF DESCRIPTION OF THE DRAWINGS/FIGURES

FIG. 1 is a table showing the clinical activity of MEDI4736 therapy in combination with tremelimumab compared to MEDI4736 monotherapy.

FIG. 2 are spider plots showing change in tumor size from baseline in selected cohorts of patients with PD-L1 negative NSCLC receiving MEDI4736 and tremelimumab (left panel) compared to those receiving MEDI4736 (10 mg/kg; CP1108) alone (right panel).

FIG. 3 are spider plots showing change in tumor size from baseline in selected cohorts of patients with PD-L1 positive NSCLC receiving MEDI4736 and tremelimumab (left panel) compared to those receiving MEDI4736 (10 mg/kg; CP1108) alone (right panel).

FIG. 4 are spider plots anchored by tremelimumab dose: 1 mg/kg (left panel), 3 mg/kg (center panel), and 10 mg/kg (right panel) showing change in tumor size from baseline in selected cohorts of patients with PD-L1 negative NSCLC receiving MEDI4736 and tremelimumab.

FIG. 5 are spider plots anchored by MEDI4736 dose: 10 mg/kg Q4W (upper left panel), 15 mg/kg (upper right panel), and 20 mg/kg (lower left panel) showing change in tumor size from baseline in selected cohorts of patients with PD-L1 negative NSCLC receiving MEDI4736 and tremelimumab.

FIGS. 6A-6D are spider plots showing change in tumor size from baseline in NSCLC patients receiving MEDI4736 and tremelimumab in FIG. 29, grouped according to NSCLC PD-L1 status: all NSCLC patients (6A); patients identified as having PD-L1⁻ NSCLC (6B); patients identified as having PD-L1⁺ NSCLC (6C); and patients with NSCLC PD-L1 status not available (6D).

FIG. 7 is a waterfall plot showing best change in tumor size from baseline in NSCLC patients receiving MEDI4736 and tremelimumab.

FIG. 8 is a waterfall plot showing best change in tumor size from baseline in NSCLC patients receiving MEDI4736 and tremelimumab in FIG. 6, identified according to PD-L1 status of the NSCLC.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “anti-PD-L1 antibody” is meant an antibody that selectively binds a PD-L1 polypeptide. Exemplary anti-PD-L1 antibodies are described for example at WO 2011/066389, which is herein incorporated by reference. MEDI4736 is an exemplary anti-PD-L1 antibody. The sequences are provided in the sequence listing below (e.g., SEQ ID NOs. 1-8).

By “negative for PD-L1” is meant that a cell or population of cells expresses undetectable or significantly reduced levels of PD-L1 relative to a PD-L1-positive cell or population of cells. In one embodiment, expression is significantly reduced when levels of PD-L1 are reduced by at least about 10%, 25%, 30% or more. In another embodiment, expression is significantly reduced when less than about 10%, 25%, 30% or more of the cells in a population (e.g., lung cancer tumor) express detectable levels of PD-L1 protein or polynucleotide. In the context of immunohistochemistry, “negative for PD-L1” means that less than about 25% of cells in a cancer sample exhibit staining for PD-L1.

By “PD-L1 polypeptide” is meant a polypeptide or fragment thereof having at least about 85% amino acid identity to NCBI Accession No. NP_{—}001254635 and having PD-1 and CD80 binding activity. PD-L1 may be used interchangeably with the term “B7-H1”).

```
PD-L1 polypeptide sequence
NCBI ACCESSION NO. NP_001254635
(SEQ ID NO: 17)
    1 mrifavfifm tywhllnapy nkinqrilvv dpvtsehelt cqaegypkae viwtssdhqv
  61 lsgkttttns kreeklfnvt stlrintttn eifyctfrrl dpeenhtael vipelplahp
121 pnerthlvil gaillclgva ltfifrlrkg rmmdvkkcgi qdtnskkqsd thleet
```

By “PD-L1 nucleic acid molecule” is meant a polynucleotide encoding a PD-L1 polypeptide. An exemplary PD-L1 nucleic acid molecule sequence is provided at NCBI Accession No. NM_{—}001267706.

```
PD-L1 nucleic acid sequence
NCBI ACCESSION NO. NM_001267706 mRNA
(SEQ ID NO: 18)
      1 ggcgcaacgc tgagcagctg gcgcgtcccg cgcggcccca gttctgcgca gcttcccgag
    61 gctccgcacc agccgcgctt ctgtccgcct gcagggcatt ccagaaagat gaggatattt
  121 gctgtcttta tattcatgac ctactggcat ttgctgaacg ccccatacaa caaaatcaac
  181 caaagaattt tggttgtgga tccagtcacc tctgaacatg aactgacatg tcaggctgag
  241 ggctacccca aggccgaagt catctggaca agcagtgacc atcaagtcct gagtggtaag
  301 accaccacca ccaattccaa gagagaggag aagcttttca atgtgaccag cacactgaga
  361 atcaacacaa caactaatga gattttctac tgcactttta ggagattaga tcctgaggaa
  421 aaccatacag ctgaattggt catcccagaa ctacctctgg cacatcctcc aaatgaaagg
  481 actcacttgg taattctggg agccatctta ttatgccttg gtgtagcact gacattcatc
  541 ttccgtttaa gaaaagggag aatgatggat gtgaaaaaat gtggcatcca agatacaaac
  601 tcaaagaagc aaagtgatac acatttggag gagacgtaat ccagcattgg aacttctgat
  661 cttcaagcag ggattctcaa cctgtggttt aggggttcat cggggctgag cgtgacaaga
  721 ggaaggaatg ggcccgtggg atgcaggcaa tgtgggactt aaaaggccca agcactgaaa
  781 atggaacctg gcgaaagcag aggaggagaa tgaagaaaga tggagtcaaa cagggagcct
  841 ggagggagac cttgatactt tcaaatgcct gaggggctca tcgacgcctg tgacagggag
  901 aaaggatact tctgaacaag gagcctccaa gcaaatcatc cattgctcat cctaggaaga
  961 cgggttgaga atccctaatt tgagggtcag ttcctgcaga agtgcccttt gcctccactc
1021 aatgcctcaa tttgttttct gcatgactga gagtctcagt gttggaacgg gacagtattt
1081 atgtatgagt ttttcctatt tattttgagt ctgtgaggtc ttcttgtcat gtgagtgtgg
1141 ttgtgaatga tttcttttga agatatattg tagtagatgt tacaattttg tcgccaaact
1201 aaacttgctg cttaatgatt tgctcacatc tagtaaaaca tggagtattt gtaaggtgct
1261 tggtctcctc tataactaca agtatacatt ggaagcataa agatcaaacc gttggttgca
1321 taggatgtca cctttattta acccattaat actctggttg acctaatctt attctcagac
1381 ctcaagtgtc tgtgcagtat ctgttccatt taaatatcag ctttacaatt atgtggtagc
1441 ctacacacat aatctcattt catcgctgta accaccctgt tgtgataacc actattattt
1501 tacccatcgt acagctgagg aagcaaacag attaagtaac ttgcccaaac cagtaaatag
1561 cagacctcag actgccaccc actgtccttt tataatacaa tttacagcta tattttactt
1621 taagcaattc ttttattcaa aaaccattta ttaagtgccc ttgcaatatc aatcgctgtg
1681 ccaggcattg aatctacaga tgtgagcaag acaaagtacc tgtcctcaag gagctcatag
1741 tataatgagg agattaacaa gaaaatgtat tattacaatt tagtccagtg tcatagcata
1801 aggatgatgc gaggggaaaa cccgagcagt gttgccaaga ggaggaaata ggccaatgtg
1861 gtctgggacg gttggatata cttaaacatc ttaataatca gagtaatttt catttacaaa
1921 gagaggtcgg tacttaaaat aaccctgaaa aataacactg gaattccttt tctagcatta
1981 tatttattcc tgatttgcct ttgccatata atctaatgct tgtttatata gtgtctggta
2041 ttgtttaaca gttctgtctt ttctatttaa atgccactaa attttaaatt catacctttc
2101 catgattcaa aattcaaaag atcccatggg agatggttgg aaaatctcca cttcatcctc
2161 caagccattc aagtttcctt tccagaagca actgctactg cctttcattc atatgttctt
2221 ctaaagatag tctacatttg gaaatgtatg ttaaaagcac gtatttttaa aatttttttc
2281 ctaaatagta acacattgta tgtctgctgt gtactttgct atttttattt attttagtgt
2341 ttcttatata gcagatggaa tgaatttgaa gttcccaggg ctgaggatcc atgccttctt
2401 tgtttctaag ttatctttcc catagctttt cattatcttt catatgatcc agtatatgtt
2461 aaatatgtcc tacatataca tttagacaac caccatttgt taagtatttg ctctaggaca
2521 gagtttggat ttgtttatgt ttgctcaaaa ggagacccat gggctctcca gggtgcactg
2581 agtcaatcta gtcctaaaaa gcaatcttat tattaactct gtatgacaga atcatgtctg
2641 gaacttttgt tttctgcttt ctgtcaagta taaacttcac tttgatgctg tacttgcaaa
2701 atcacatttt ctttctggaa attccggcag tgtaccttga ctgctagcta ccctgtgcca
2761 gaaaagcctc attcgttgtg cttgaaccct tgaatgccac cagctgtcat cactacacag
2821 ccctcctaag aggcttcctg gaggtttcga gattcagatg ccctgggaga tcccagagtt
2881 tcctttccct cttggccata ttctggtgtc aatgacaagg agtaccttgg ctttgccaca
2941 tgtcaaggct gaagaaacag tgtctccaac agagctcctt gtgttatctg tttgtacatg
3001 tgcatttgta cagtaattgg tgtgacagtg ttctttgtgt gaattacagg caagaattgt
3061 ggctgagcaa ggcacatagt ctactcagtc tattcctaag tcctaactcc tccttgtggt
3121 gttggatttg taaggcactt tatccctttt gtctcatgtt tcatcgtaaa tggcataggc
3181 agagatgata cctaattctg catttgattg tcactttttg tacctgcatt aatttaataa
3241 aatattctta tttattttgt tacttggtac accagcatgt ccattttctt gtttattttg
3301 tgtttaataa aatgttcagt ttaacatccc agtggagaaa gttaaaaaa
```

By “an anti-CTLA4 antibody” is meant an antibody that selectively binds a CTLA4 polypeptide. Exemplary anti-CTLA4 antibodies are described for example at U.S. Pat. Nos. 6,682,736; 7,109,003; 7,123,281; 7,411,057; 7,824,679; 8,143,379; 7,807,797; and 8,491,895 (Tremelimumab is 11.2.1, therein), which are herein incorporated by reference. Tremelimumab is an exemplary anti-CTLA4 antibody. Tremelimumab sequences are provided in the sequence listing below.

The term “antibody,” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind PD-L1 specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)2 fragment in which the two arms of the antibody molecule remain linked and comprise two-antigen binding sites. The F(ab′)2 fragment has the ability to cros slink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

By “biologic sample” is meant any tissue, cell, fluid, or other material derived from an organism. In one embodiment, a biological sample is a lung cancer tumor biopsy sample.

A “biomarker” or “marker” as used herein generally refers to a protein, nucleic acid molecule, clinical indicator, or other analyte that is associated with a disease. In one embodiment, a marker is differentially present in a biological sample obtained from a subject having a disease (e.g., lung cancer) relative to the level present in a control sample or reference.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “disease” is meant any condition or disorder that damages, interferes with or dysregulates the normal function of a cell, tissue, or organ. In a disease such as cancer (e.g., lung cancer) the normal function of a cell tissue or organ is subverted to enable immune evasion and/or escape. Lung cancer includes small cell lung cancer (SCLC) and non-small cell lung cancer (NSCLC). There are three main subtypes of NSCLC: squamous cell carcinoma, adenocarcinoma, and large cell (undifferentiated) carcinoma. Other subtypes include adenosquamous carcinoma and sarcomatoid carcinoma.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “reference” is meant a standard of comparison.

By “responsive” in the context of therapy is meant susceptible to treatment.

By “specifically binds” is meant a compound (e.g., antibody) that recognizes and binds a molecule (e.g., polypeptide), but which does not substantially recognize and bind other molecules in a sample, for example, a biological sample. For example, two molecules that specifically bind form a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant K_{A} is higher than 10⁶M⁻¹, or more preferably higher than 10⁸M⁻¹. If necessary, non-specific binding can be reduced without substantially affecting specific binding by varying the binding conditions. The appropriate binding conditions such as concentration of antibodies, ionic strength of the solution, temperature, time allowed for binding, concentration of a blocking agent (e.g., serum albumin, milk casein), etc., may be optimized by a skilled artisan using routine techniques.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION OF THE INVENTION

As described below, the present invention features methods of treating lung cancer (e.g., non-small cell lung cancer) with an anti-PD-L1 antibody and tremelimumab in a subject identified as having a PD-L1 negative tumor.

### CTLA4 and PD-L1

The role of the immune system, in particular T cell-mediated cytotoxicity, in tumor control is well recognized. There is mounting evidence that T cells control tumor growth and survival in cancer patients, both in early and late stages of the disease. However, tumor-specific T-cell responses are difficult to mount and sustain in cancer patients.

Two T cell modulatory pathways receiving significant attention to date signal through cytotoxic T lymphocyte antigen-4 (CTLA-4, CD152) and programmed death ligand 1 (PD-L1, also known as B7H-1 or CD274).

CTLA4 is expressed on activated T cells and serves as a co-inhibitor to keep T cell responses in check following CD28-mediated T cell activation. CTLA4 is believed to regulate the amplitude of the early activation of naïve and memory T cells following TCR engagement and to be part of a central inhibitory pathway that affects both antitumor immunity and autoimmunity. CTLA4 is expressed primarily on T cells, and the expression of its ligands CD80 (B7.1) and CD86 (B7.2), is largely restricted to antigen-presenting cells, T cells, and other immune mediating cells. Antagonistic anti-CTLA4 antibodies that block the CTLA4 signaling pathway have been reported to enhance T cell activation. One such antibody, ipilimumab, was approved by the FDA in 2011 for the treatment of metastatic melanoma. Another anti-CTLA4 antibody, tremelimumab, was tested in phase III trials for the treatment of advanced melanoma but did not significantly increase the overall survival of patients compared to the standard of care (temozolomide or dacarbazine) at that time.

PD-L1 is also part of a complex system of receptors and ligands that are involved in controlling T cell activation. In normal tissue, PD-L1 is expressed on T cells, B cells, dendritic cells, macrophages, mesenchymal stem cells, bone marrow-derived mast cells, as well as various nonhematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between PD-L1 and its receptors are able to relieve PD-L1-dependent immunosuppressive effects and enhance the cytotoxic activity of antitumor T cells in vitro.

### Anti-PD-L1 Antibodies

Antibodies that specifically bind and inhibit PD-L1 activity (e.g., binding to PD-1 and/or CD80) are useful for the treatment of lung cancer (e.g., non-small cell lung cancer).

MEDI4736 is an exemplary anti-PD-L1 antibody that is selective for a PD-L1 polypeptide and blocks the binding of PD-L1 to the PD-1 and CD80 receptors. MEDI4736 can relieve PD-L1 -mediated suppression of human T-cell activation in vitro and inhibits tumor growth in a xenograft model via a T-cell dependent mechanism.

Information regarding MEDI4736 (or fragments thereof) for use in the methods provided herein can be found in U.S. Pat. No. 8,779,108, the disclosure of which is incorporated herein by reference in its entirety. The fragment crystallizable (Fc) domain of MEDI4736 contains a triple mutation in the constant domain of the IgG1 heavy chain that reduces binding to the complement component C1q and the Fcγ receptors responsible for mediating antibody-dependent cell-mediated cytotoxicity (ADCC).

MEDI4736 and antigen-binding fragments thereof for use in the methods provided herein comprises a heavy chain and a light chain or a heavy chain variable region and a light chain variable region. In a specific aspect, MEDI4736 or an antigen-binding fragment thereof for use in the methods provided herein comprises a light chain variable region comprising the amino acid sequence of SEQ ID NO:1 and a heavy chain variable region comprising the amino acid sequence of SEQ ID NO:2. In a specific aspect, MEDI4736 or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs:3-5, and wherein the light chain variable region comprises the Kabat-defined CDR1, CDR2, and CDR3 sequences of SEQ ID NOs:6-8. Those of ordinary skill in the art would easily be able to identify Chothia-defined, Abm-defined or other CDR definitions known to those of ordinary skill in the art. In a specific aspect, MEDI4736 or an antigen-binding fragment thereof for use in the methods provided herein comprises the variable heavy chain and variable light chain CDR sequences of the 2.14H9OPT antibody as disclosed in WO 2011/066389 A1, which is herein incorporated by reference in its entirety.

### Selection of Tremelimumab and Anti-PD-L1 Treatment

Subjects suffering from lung cancer (e.g., non-small cell lung cancer) may be tested for PD-L1 polynucleotide or polypeptide expression in the course of selecting a treatment method. Patients identified as having tumors that are negative for PD-L1 (e.g., as defined by Ct or IHC-M score) or by having reduced or undetectable levels of PD-L1 relative to a reference level are identified as responsive to treatment with a combination of an anti-PD-L1 antibody and tremelimumab. Such patients are administered an anti-PD-L1 antibody, such as MEDI4736, or an antigen-binding fragment thereof in combination with tremelimumab.

Information regarding tremelimumab (or antigen-binding fragments thereof) for use in the methods provided herein can be found in U.S. Pat. No. 6,682,736 (where it is referred to as 11.2.1), the disclosure of which is incorporated herein by reference in its entirety. Tremelimumab (also known as CP-675,206, CP-675, CP-675206, and ticilimumab) is a human IgG₂ monoclonal antibody that is highly selective for CTLA4 and blocks binding of CTLA4 to CD80 (B7.1) and CD86 (B7.2). It has been shown to result in immune activation in vitro and some patients treated with tremelimumab have shown tumor regression.

Tremelimumab for use in the methods provided herein comprises a heavy chain and a light chain or a heavy chain variable region and a light chain variable region. In a specific aspect, tremelimumab or an antigen-binding fragment thereof for use in the methods provided herein comprises a light chain variable region comprising the amino acid sequence of SEQ ID NO:9 and a heavy chain variable region comprising the amino acid sequence of SEQ ID NO:10. In a specific aspect, tremelimumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs:11-13, and wherein the light chain variable region comprises the Kabat-defined CDR1, CDR2, and CDR3 sequences of SEQ ID NOs:14-16. Those of ordinary skill in the art would easily be able to identify Chothia-defined, Abm-defined or other CDR definitions known to those of ordinary skill in the art. In a specific aspect, tremelimumab or an antigen-binding fragment thereof for use in the methods provided herein comprises the variable heavy chain and variable light chain CDR sequences of the 11.2.1 antibody as disclosed in U.S. Pat. No. 6,682,736, which is herein incorporated by reference in its entirety.

In certain aspects, a patient presenting with a solid lung cancer tumor is administered MEDI4736 or an antigen-binding fragment thereof and tremelimumab or an antigen-binding fragment thereof. MEDI4736 or an antigen-binding fragment thereof and tremelimumab or an antigen-binding fragment thereof can be administered only once or infrequently while still providing benefit to the patient. In further aspects the patient is administered additional follow-on doses. Follow-on doses can be administered at various time intervals depending on the patient's age, weight, clinical assessment, tumor burden, and/or other factors, including the judgment of the attending physician.

The intervals between doses of MEDI4736 or an antigen-binding fragment thereof can be every two or three weeks. The intervals between doses of tremelimumab or an antigen-binding fragment thereof can be every four weeks. The intervals between doses of tremelimumab or an antigen-binding fragment thereof can be every twelve weeks. The intervals between doses of tremelimumab or an antigen-binding fragment thereof can be every four weeks for six cycles and then every twelve weeks. In certain aspects, MEDI4736 or an antigen-binding fragment thereof is administered about twice as frequently as tremelimumab or an antigen-binding fragment thereof. In certain aspects, MEDI4736 or an antigen-binding fragment thereof is administered about six times as frequently as tremelimumab or an antigen-binding fragment thereof.

In some embodiments, at least two doses of MEDI4736 or an antigen-binding fragment thereof and tremelimumab or an antigen-binding fragment thereof are administered to the patient. In some embodiments, at least three doses, at least four doses, at least five doses, at least six doses, at least seven doses, at least eight doses, at least nine doses, at least ten doses, or at least fifteen doses or more can be administered to the patient. In some embodiments, MEDI4736 or an antigen-binding fragment thereof is administered over a two-week treatment period, over a four-week treatment period, over a six-week treatment period, over an eight-week treatment period, over a twelve-week treatment period, over a twenty-four-week treatment period, or over a one-year or more treatment period. In some embodiments, tremelimumab or an antigen-binding fragment thereof is administered over a four-week treatment period, over an eight-week treatment period, over a twelve-week treatment period, over a sixteen-week treatment period, over a twenty-week treatment period, over a twenty-four-week treatment period, over a thirty-six-week treatment period, over a forty-eight-week treatment period, or over a one-year or more treatment period

The amount of MEDI4736 or an antigen-binding fragment thereof and the amount of tremelimumab or an antigen-binding fragment thereof to be administered to the patient will depend on various parameters such as the patient's age, weight, clinical assessment, tumor burden and/or other factors, including the judgment of the attending physician.

In certain aspects the patient is administered one or more doses of MEDI4736 or an antigen-binding fragment thereof wherein the dose is about 0.3 mg/kg. In certain aspects the patient is administered one or more doses of MEDI4736 or an antigen-binding fragment thereof wherein the dose is about 1 mg/kg. In certain aspects the patient is administered one or more doses of MEDI4736 or an antigen-binding fragment thereof wherein the dose is about 3 mg/kg. In certain aspects the patient is administered one or more doses of MEDI4736 or an antigen-binding fragment thereof wherein the dose is about 10 mg/kg.

In certain aspects the patient is administered at least two doses of MEDI4736 or an antigen-binding fragment thereof wherein the dose is about 0.3 mg/kg. In certain aspects the patient is administered at least two doses of MEDI4736 or an antigen-binding fragment thereof wherein the dose is about 1 mg/kg. In certain aspects the patient is administered at least two doses of MEDI4736 or an antigen-binding fragment thereof wherein the dose is about 3 mg/kg. In certain aspects the patient is administered at least two doses of MEDI4736 or an antigen-binding fragment thereof wherein the dose is about 10 mg/kg. In some embodiments, the at least two doses are administered about two weeks apart.

In certain aspects the patient is administered at least three doses of MEDI4736 or an antigen-binding fragment thereof wherein the dose is about 0.3 mg/kg. In certain aspects the patient is administered at least three doses of MEDI4736 or an antigen-binding fragment thereof wherein the dose is about 1 mg/kg. In certain aspects the patient is administered at least three doses of MEDI4736 or an antigen-binding fragment thereof wherein the dose is about 3 mg/kg. In certain aspects the patient is administered at least three doses of MEDI4736 or an antigen-binding fragment thereof wherein the dose is about 10 mg/kg. In some embodiments, the at least three doses are administered about two weeks apart.

In certain aspects the patient is administered one or more doses of tremelimumab or an antigen-binding fragment thereof wherein the dose is about 1 mg/kg. In certain aspects the patient is administered one or more doses of tremelimumab or an antigen-binding fragment thereof wherein the dose is about 3 mg/kg. In certain aspects the patient is administered one or more doses of tremelimumab or an antigen-binding fragment thereof wherein the dose is about 10 mg/kg.

In certain aspects the patient is administered at least two doses of tremelimumab or an antigen-binding fragment thereof wherein the dose is about 1 mg/kg. In certain aspects the patient is administered at least two doses of tremelimumab or an antigen-binding fragment thereof wherein the dose is about 3 mg/kg. In certain aspects the patient is administered at least two doses of tremelimumab or an antigen-binding fragment thereof wherein the dose is about 10 mg/kg. In some embodiments, the at least two doses are administered about four weeks apart. In some embodiments, the at least two doses are administered about twelve weeks apart.

In certain aspects the patient is administered at least three doses of tremelimumab or an antigen-binding fragment thereof wherein the dose is about 1 mg/kg. In certain aspects the patient is administered at least three doses of tremelimumab or an antigen-binding fragment thereof wherein the dose is about 3 mg/kg. In certain aspects the patient is administered at least three doses of tremelimumab or an antigen-binding fragment thereof wherein the dose is about 10 mg/kg. In some embodiments, the at least three doses are administered about four weeks apart. In some embodiments, the at least three doses are administered about twelve weeks apart.

In certain aspects, administration of MEDI4736 or an antigen-binding fragment thereof and/or tremelimumab or an antigen-binding fragment according to the methods provided herein is through parenteral administration. For example, MEDI4736 or an antigen-binding fragment thereof and/or tremelimumab or an antigen-binding fragment can be administered by intravenous infusion or by subcutaneous injection. In some embodiments, the administration is by intravenous infusion.

In certain aspects, 0.3 mg/kg of MEDI4736 or an antigen-binding fragment thereof and 1 mg/kg of tremelimumab or an antigen-binding fragment thereof are administered to a patient. In certain aspects, 0.3 mg/kg of MEDI4736 or an antigen-binding fragment thereof and 3 mg/kg of tremelimumab or an antigen-binding fragment thereof are administered to a patient. In certain aspects, 0.3 mg/kg of MEDI4736 or an antigen-binding fragment thereof and 10 mg/kg of tremelimumab or an antigen-binding fragment thereof are administered to a patient.

In certain aspects, 1 mg/kg of MEDI4736 or an antigen-binding fragment thereof and 1 mg/kg of tremelimumab or an antigen-binding fragment thereof are administered to a patient. In certain aspects, 1 mg/kg of MEDI4736 or an antigen-binding fragment thereof and 3 mg/kg of tremelimumab or an antigen-binding fragment thereof are administered to a patient. In certain aspects, 1 mg/kg of MEDI4736 or an antigen-binding fragment thereof and 10 mg/kg of tremelimumab or an antigen-binding fragment thereof are administered to a patient.

In certain aspects, 3 mg/kg of MEDI4736 or an antigen-binding fragment thereof and 1 mg/kg of tremelimumab or an antigen-binding fragment thereof are administered to a patient. In certain aspects, 3 mg/kg of MEDI4736 or an antigen-binding fragment thereof and 3 mg/kg of tremelimumab or an antigen-binding fragment thereof are administered to a patient. In certain aspects, 3 mg/kg of MEDI4736 or an antigen-binding fragment thereof and 10 mg/kg of tremelimumab or an antigen-binding fragment thereof are administered to a patient.

In certain aspects, 10 mg/kg of MEDI4736 or an antigen-binding fragment thereof and 1 mg/kg of tremelimumab or an antigen-binding fragment thereof are administered to a patient. In certain aspects, 10 mg/kg of MEDI4736 or an antigen-binding fragment thereof and 3 mg/kg of tremelimumab or an antigen-binding fragment thereof are administered to a patient. In certain aspects, 10 mg/kg of MEDI4736 or an antigen-binding fragment thereof and 10 mg/kg of tremelimumab or an antigen-binding fragment thereof are administered to a patient.

The methods provided herein can decrease or retard lung cancer tumor growth. In some aspects the reduction or retardation can be statistically significant. A reduction in lung cancer tumor growth can be measured by comparison to the growth of patient's tumor at baseline, against an expected tumor growth, against an expected tumor growth based on a large patient population, or against the tumor growth of a control population.

In certain aspects, a tumor response is measured using the Immune-related Response Criteria (irRc). In certain aspects, a tumor response is measured using the Response Evaluation Critera in Solid Tumors (RECIST).

In certain aspects, a tumor response is detectable at week 7. In certain aspects, a tumor response is detectable at week 13. In certain aspects, a tumor response is detectable at week 17. In certain aspects, a tumor response is detectable at week 25. In certain aspects, a tumor response is detectable at week 33. In certain aspects, a tumor response is detectable at week 41. In certain aspects, a tumor response is detectable at week 49. In certain aspects, a tumor response is detectable at week 64.

In certain aspects, a tumor response is detectable after administration of three doses of MEDI4736 or an antigen-binding fragment thereof and two doses of tremelimumab or an antigen-binding fragment thereof. In certain aspects, a tumor response is detectable after administration of six doses of MEDI4736 or an antigen-binding fragment thereof and three doses of tremelimumab or an antigen-binding fragment thereof. In certain aspects, a tumor response is detectable after administration of eight doses of MEDI4736 or an antigen-binding fragment thereof and four doses of tremelimumab or an antigen-binding fragment thereof. In certain aspects, a tumor response is detectable after administration of twelve doses of MEDI4736 or an antigen-binding fragment thereof and six doses of tremelimumab or an antigen-binding fragment thereof. In certain aspects, a tumor response is detectable after administration of sixteen doses of MEDI4736 or an antigen-binding fragment thereof and seven doses of tremelimumab or an antigen-binding fragment thereof. In certain aspects, a tumor response is detectable after administration of twenty doses of MEDI4736 or an antigen-binding fragment thereof and eight doses of tremelimumab or an antigen-binding fragment thereof. In certain aspects, a tumor response is detectable after administration of twenty-four doses of MEDI4736 or an antigen-binding fragment thereof and eight doses of tremelimumab or an antigen-binding fragment thereof. In certain aspects, a tumor response is detectable after administration of twenty-six doses of MEDI4736 or an antigen-binding fragment thereof and nine doses of tremelimumab or an antigen-binding fragment thereof.

In certain aspects, a patient achieves disease control (DC). Disease control can be a complete response (CR), partial response (PR), or stable disease (SD).

A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR.

A “partial response” (PR) refers to a decrease in tumor burden ≧30% relative to baseline. Confirmation can be obtained using a consecutive repeat assessment at least 4 weeks from the date of first documentation.

“Stable disease” (SD) indicates a decrease in tumor burden of less than about 30% relative to baseline cannot be established and a 20% or greater increase compared to nadir cannot be established.

In certain aspects, administration of MEDI4736 or an antigen-binding fragment thereof and tremelimumab or an antigen-binding fragment thereof can increase progression-free survival (PFS).

In certain aspects, administration of MEDI4736 or an antigen-binding fragment thereof and tremelimumab or an antigen-binding fragment thereof can increase overall survival (OS).

In some embodiments, the patient has previously received treatment with at least one chemotherapeutic agent. In some embodiments, the patient has previously received treatment with at least two chemotherapeutic agents. The chemotherapeutic agent can be, for example, and without limitation, Vemurafenib, Erlotinib, Afatinib, Cetuximab, Carboplatin, Bevacizumab, Erlotinib, Gefitinib, and/or Pemetrexed.

In some embodiments, the lung cancer tumor is refractory or resistant to at least one chemotherapeutic agent. In some embodiments, the tumor is refractory or resistant to at least two chemotherapeutic agents. The tumor can be refractory or resistant to one or more of, for example, and without limitation, Vemurafenib, Erlotinib, Afatinib, Cetuximab, Carboplatin, Bevacizumab, Erlotinib, Gefitinib, and/or Pemetrexed.

In some embodiments, the patient has an Eastern Cooperative Oncology Group (ECOG) (Oken M M, et al. Am. J. Clin. Oncol. 5: 649-55 (1982)) performance status of 0, 1, or 2 prior to the administration of MEDI4736 or an antigen-binding fragment thereof and tremelimumab or an antigen-binding fragment thereof.

As provided herein, MEDI4736 or an antigen-binding fragment thereof can also decrease free (soluble) PD-L1 levels. Free (soluble) PD-L1 refers to PD-L1 that is not bound (e.g., by MEDI4736). In some embodiments, sPD-L1 levels are reduced and/or undetectable following administration of MEDI4736 or an antigen-binding fragment thereof and tremelimumab or an antigen-binding fragment thereof. In some embodiments, administration of MEDI4736 or an antigen-binding fragment thereof and tremelimumab or an antigen-binding fragment thereof reduces the rate of increase of free (soluble) PD-L1 levels as compared, e.g., to the rate of increase of free (soluble) PD-L1 levels prior to the administrations.

Treatment of a patient with a solid lung cancer tumor using both MEDI4736 or an antigen-binding fragment thereof and tremelimumab or an antigen-binding fragment thereof (i.e., co-therapy) as provided herein can result in an additive and/or synergistic effect. As used herein, the term “synergistic” refers to a combination of therapies (e.g., a combination of MEDI4736 or an antigen-binding fragment thereof and tremelimumab or an antigen-binding fragment thereof) which is more effective than the additive effects of the single therapies.

A synergistic effect of a combination of therapies (e.g., a combination of a MEDI4736 or an antigen-binding fragment thereof and tremelimumab or an antigen-binding fragment thereof) permits the use of lower dosages of one or more of the therapeutic agents and/or less frequent administration of said therapeutic agents to a patient with a solid lung cancer tumor. The ability to utilize lower dosages of therapeutic agents and/or to administer said therapies less frequently reduces the toxicity associated with the administration of said therapies to a subject without reducing the efficacy of said therapies in the treatment of a solid lung cancer tumor. In addition, a synergistic effect can result in improved efficacy of therapeutic agents in the management, treatment, or amelioration of an solid lung cancer tumor. The synergistic effect of a combination of therapeutic agents can avoid or reduce adverse or unwanted side effects associated with the use of either single therapy.

In co-therapy, MEDI4736 or an antigen-binding fragment thereof can be optionally included in the same pharmaceutical composition as the tremelimumab or an antigen-binding fragment thereof, or may be included in a separate pharmaceutical composition. In this latter case, the pharmaceutical composition comprising MEDI4736 or an antigen-binding fragment thereof is suitable for administration prior to, simultaneously with, or following administration of the pharmaceutical composition comprising tremelimumab or an antigen-binding fragment thereof. In certain instances, the MEDI4736 or an antigen-binding fragment thereof is administered at overlapping times as tremelimumab or an antigen-binding fragment thereof in a separate composition.

### Kits

The invention provides kits for characterizing a lung cancer tumor sample for PD-L1 in combination with a therapeutic composition comprising an anti-PD-L1 antibody, such as MEDI4736, or an antigen-binding fragment thereof and tremelimumab. In one embodiment, the kit includes a therapeutic composition comprising MEDI4736 and tremelimumab, each in unit dosage form.

A diagnostic kit of the invention provides a reagent (e.g., an antibody or antigen binding fragment thereof that selectively bind a PD-L1 polypeptide) for measuring relative expression and/or localization of a PD-L1 polypeptide. In other embodiments, the kit further includes reagents suitable for PD-L1 immunohistochemistry.

In some embodiments, the kit comprises a sterile container which contains a therapeutic and/or diagnostic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, the kit further comprises instructions for measuring PD-L1 polypeptide expression and/or instructions for administering the anti-PD-L1 antibody and tremelimumab to a subject having a lung cancer (e.g., non-small cell lung cancer) selected as negative for PD-L1. In particular embodiments, the instructions include at least one of the following: description of the therapeutic agent; dosage schedule and administration for treatment or prevention of lung cancer (e.g., non-small cell lung cancer) or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry immunohistochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Non-Small Cell Lung Cancer Trial

Subjects in this study are required to be 18 years of age or older and have histologically- or cytologically-confirmed non-small cell lung cancer (NSCLC; squamous and non-squamous), with at least one measurable lesion according to Response Evaluation Criteria in Solid Tumors (RECIST) guidelines v1.1, which is herein incorporated by reference in its entirety.

The subjects are also required to have failed to respond to standard treatment, relapsed following standard treatment, declined standard treatment, or have not been eligible for standard treatment. Subjects will have an Eastern Cooperative Oncology Group (ECOG) performance status of 0-1.

The subjects are also required to have adequate organ (hepatic and renal) and marrow function. Adequate organ and marrow function are defined as: hemoglobin≧9 g/dL; absolute neutrophil count≧1,500/mm³; platelet count≧100,000/mm³; total bilirubin≦1.5×upper limit of normal (ULN), unless associated with Gilbert's syndrome or liver metastasis (for these subjects, baseline total bilirubin must be ≦3.0 mg/dL); alanine aminotransferase (ALT) and aspartate aminotransferase (AST) must be ≦2.5×ULN unless associated with hepatic metastases (for these subjects, ALT and AST must be ≦5×ULN); and serum creatinine≦2.0 mg/dL.

Subjects are not able to participate if they are on any concurrent chemotherapy, immunotherapy, biologic, or hormonal therapy for cancer treatment. Subjects are not able to participate if they have taken any investigational anticancer therapy within 28 days prior to the first dose of MEDI4736 and tremelimumab. Subjects are not able to participate if they have any prior Grade≧3 immune-related adverse event (irAE) while receiving immunotherapy, including anti-CTLA-4 treatment, or any unresolved irAE>Grade 1. Subjects are also not able to participate if they have undergone a major surgical procedure (as defined by the investigator) within 28 days prior to the first dose of MEDI4736 and tremelimumab or if they are still recovering from prior surgery. Subjects are also not able to participate if they have unresolved toxicities from prior anticancer therapy, defined as having not resolved to National Cancer Institute Common Terminology Criteria for Adverse Events (NCI CTCAE) v4.03 Grade 0 or 1 with the exception of alopecia and laboratory values listed per the inclusion criteria. Subjects with irreversible toxicity that is not reasonably expected to be exacerbated by MEDI4736 and tremelimumab may be included. Subjects are also excluded if they are currently using, or have used immunosuppressive medication within 14 days before the first dose of MEDI4736 and tremelimumab with the exceptions of intranasal and inhaled corticosteroids or systemic corticosteroids at physiologic doses not to exceed 10 mg/day of prednisone or equivalent.

Subjects are not able to participate if they have active or prior autoimmune disease, including inflammatory bowel disease, diverticulitis, irritable bowel disease, celiac disease, Wegener syndrome, and Hashimoto syndrome, within the past 3 years, except for vitiligo, alopecia, Grave's disease, or psoriasis not requiring systemic treatment (within the past 3 years). Subjects are also not able to participate if they have a history of primary immunodeficiency or tuberculosis, if they have known active or chronic viral hepatitis A, B, or C; if they have human immunodeficiency virus (HIV); other active serious illnesses or uncontrolled inter-current illnesses; have received live, attenuated vaccine within 28 days prior to the first dose of MEDI4736 and tremelimumab; have other invasive malignancy within 5 years; or known allergy or hypersensitivity to study drug formulations.

### Example 2

### Design of the Study

The study is an open-label Phase 1b study of the combination of anti-PD-L1 antibody (MEDI4736) and tremelimumab.

Twelve subjects were treated with 3 subjects each in Cohort 1a (1 mg/kg tremelimumab and 3 mg/kg MEDI4736), Cohort 2a (1 mg/kg tremelimumab and 10 mg/kg MEDI4736), Cohort 3a (1 mg/kg tremelimumab and 15 mg/kg MEDI4736), and Cohort 3b (3 mg/kg tremelimumab and 10 mg/kg MEDI4736). Two subjects in Cohort la (one subject withdrew consent after 2 doses of both agents) completed approximately 115 days of follow-up; Cohort 2a subjects completed approximately 56 days of follow-up; and subjects in Cohorts 3a and 3b completed 28 days of follow up.

Baseline levels of PDL-1 tumor expression data for 7 subjects on the study are provided in Table 1 (below). Additional information is provided in Table 2.

| | | PD-L1 Result | BOR (cut-off | Best Change in |
|---|---|---|---|---|
| Cohort | M Score | (@25% M | Jun. 4, 2014) | target Lesion |
| Cohort 5 (15 MEDI4736/10 Treme) | 13 | NEG | No Assessments | NA |
| Cohort 3a (15 MEDI4736/1 Treme) | 0 | NEG | Unconfirmed PR | 65.2% Decrease |
| Cohort 2a (10 MEDI4736/1 Treme) | 0 | NEG | PD | 3.7% Increase |
| Cohort 2a (10 MEDI4736/1 Treme) | 7 | NEG | SD | 1.6% Increase |
| Cohort 3a (15 MEDI4736/1 Treme) | 2 | NEG | SD | 26.7% |
| | | | | Decrease |
| Cohort 3b (10 MEDI4736/3 Treme) | 2 | NEG | Unconfirmed PR | 38.9% Decrease |
| Cohort 3b (10 MEDI4736/3 Treme) | 0 | NEG | PD | 23.5% Increase |

TABLE 2

| | A | B | C | D | E | F | G | H | I | J | L | M |
|---|---|---|---|---|---|---|---|---|---|---|---|---|
| | | | | | | | PD-L1 | | | | Best | |
| | | | 3+ | 2+ | 1+ | | Result | | | BOR | Change | |
| | | | Tumor | Tumor | Tumor | | (@25% | | COLLEC- | (cut-off | in | |
| | | | Mem- | Mem- | Mem- | M | M Score | | TION | Jun. 4, | target | Current |
| 1 | Cohort | SID | brane | brane | brane | Score | Cutoff) | REC'D | DATE | 2014) | Lesion | Status |
| 2 | Cohort | 2000060020 | 0 | 8 | 5 | 13 | NEG | 16 Jun. 2014 | 8 Apr. 2014 | No | NA | On treatment. |
| | 5 (15 | | | | | | | | | Assess- | | Waiting for |
| | MEDI4736/ | | | | | | | | | ments | | week 8 scans |
| | 10 Treme) | | | | | | | | | | | |
| 3 | Cohort | 2000060009 | 0 | 0 | 0 | 0 | NEG | 16 Jun. 2014 | 8 Jan. 2014 | Uncon- | 65.2% | On treatment. |
| | 3a (15 | | | | | | | | | firmed | Decrease | Waiting for |
| | MEDI4736/ | | | | | | | | | PR | | week 16 scans |
| | 1 Treme) | | | | | | | | | | | |
| 4 | Cohort | 2000060004 | 0 | 0 | 0 | 0 | NEG | 16 Jun. 2014 | 25 May 2012 | PD | 3.7% | On treatment |
| | 2a (10 | | | | | | | | | | Increase | past progres- |
| | MEDI4736/ | | | | | | | | | | | sion at week |
| | 1 Treme) | | | | | | | | | | | 20. |
| 5 | Cohort | 2000062007 | 0 | 5 | 2 | 7 | NEG | 16 Jun. 2014 | 31 Aug. 2011 | SD | 1.6% | On treatment |
| | 2a (10 | | | | | | | | | | Increase | at week 20. |
| | MEDI4736/ | | | | | | | | | | | |
| | 1 Treme) | | | | | | | | | | | |
| 6 | Cohort | 2000060014 | 0 | 2 | 0 | 2 | NEG | 16 Jun. 2014 | 12 Jun. 2007 | SD | 26.7% | Off treatment |
| | 3a (15 | | | | | | | | | | Decrease | at 8 weeks |
| | MEDI4736/ | | | | | | | | | | | (colitis) |
| | 1 Treme) | | | | | | | | | | | |
| 7 | Cohort | 2000062015 | 0 | 1 | 1 | 2 | NEG | 16 Jun. 2014 | 4 Oct. 2013 | Uncon- | 38.9% | On treatment |
| | 3b (10 | | | | | | | | | firmed | Decrease | at 12 weeks. |
| | MEDI4736/ | | | | | | | | | PR | | |
| | 3 Treme) | | | | | | | | | | | |
| 8 | Cohort | 2000060010 | 0 | 0 | 0 | 0 | NEG | 16 Jun. 2014 | 1 Aug. 2013 | PD | 23.5% | Off treatment |
| | 3b (10 | | | | | | | | | | Increase | at 8 weeks |
| | MEDI4736/ | | | | | | | | | | | (colitis, PD) |
| | 3 Treme) | | | | | | | | | | | |

Subject tissue of NSCLC patients was characterized for PDL1 expression by immunohistochemistry in formalin fixed and paraffin embedded tissue samples. A sample was determined to be “PD-L1 positive” if the sample contained 25% or more tumor cells with PDL1 membrane staining. This is expressed as immunohistochemistry membrane (M)-score. All samples were scored as “negative” for PDL-1 expression. Tumor assessments are available on 6 of 7 patients. Three patients treated with a combination of tremelimumab and MEDI4736.

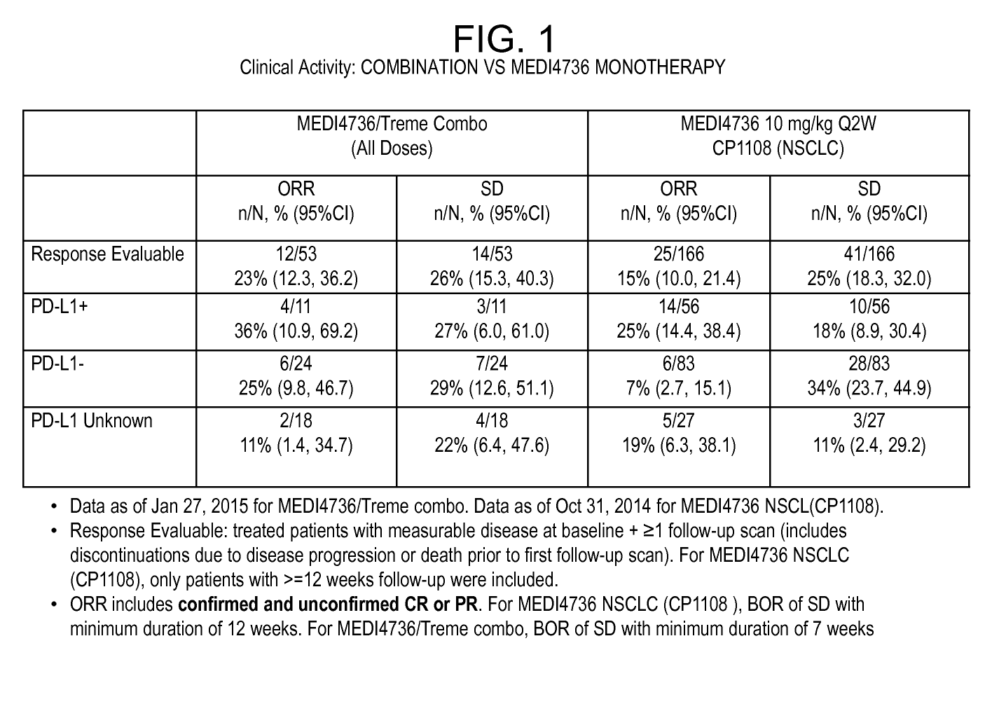

Clinical activity in NSCLC was observed with treatment with MEDI4736 and tremelimumab (all doses) showed increases in overall response rate, compared to treatment with MEDI4736 monotherapy (10 mg/kg Q2W (FIG. 1). Response was evaluable in treated patients with measurable disease at baseline+≧1 follow-up scan (includes discontinuations due to disease progression or death prior to first follow-up scan). For MEDI4736 NSCLC (CP1108), only patients with 12 weeks follow-up were included. Overall response rate (ORR) includes confirmed and unconfirmed complete response (CR) or partial response (PR). For MEDI4736 NSCLC monotherapy (CP1108, 10 mg/kg Q2W), best overall response (BOR) of stable disease (SD) with minimum duration of 12 weeks is presented. For the combination of MEDI4736 and tremelimumab, BOR of SD with minimum duration of 7 weeks is presented.

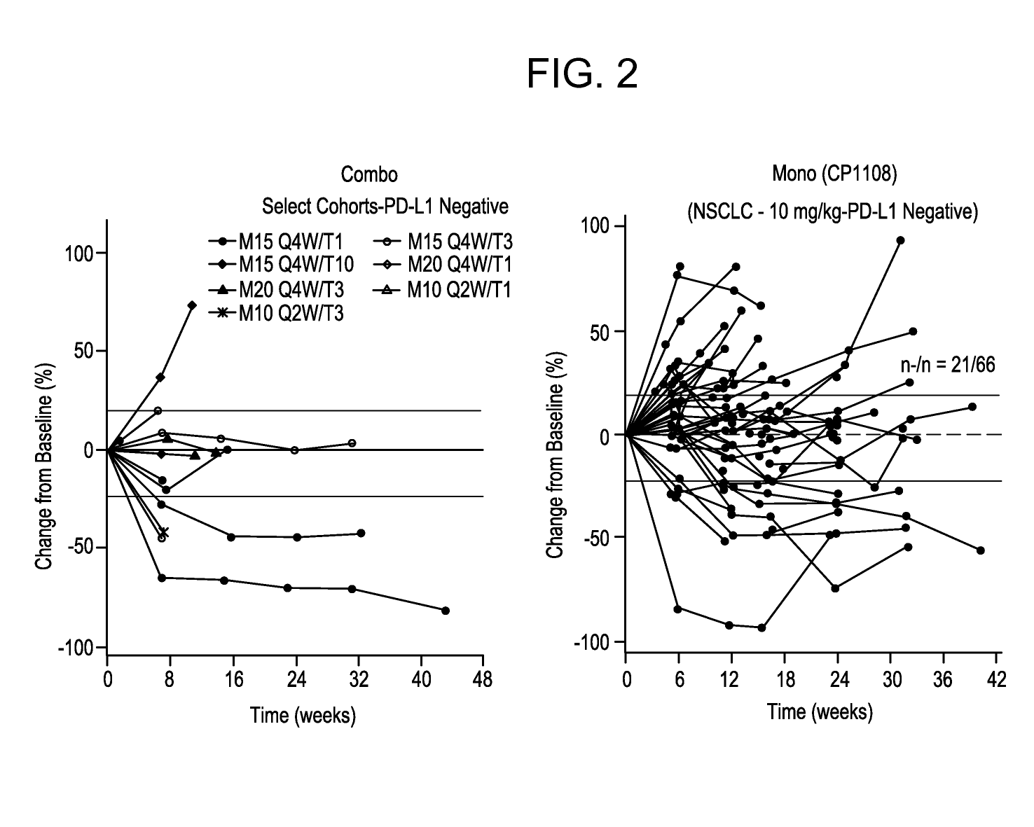

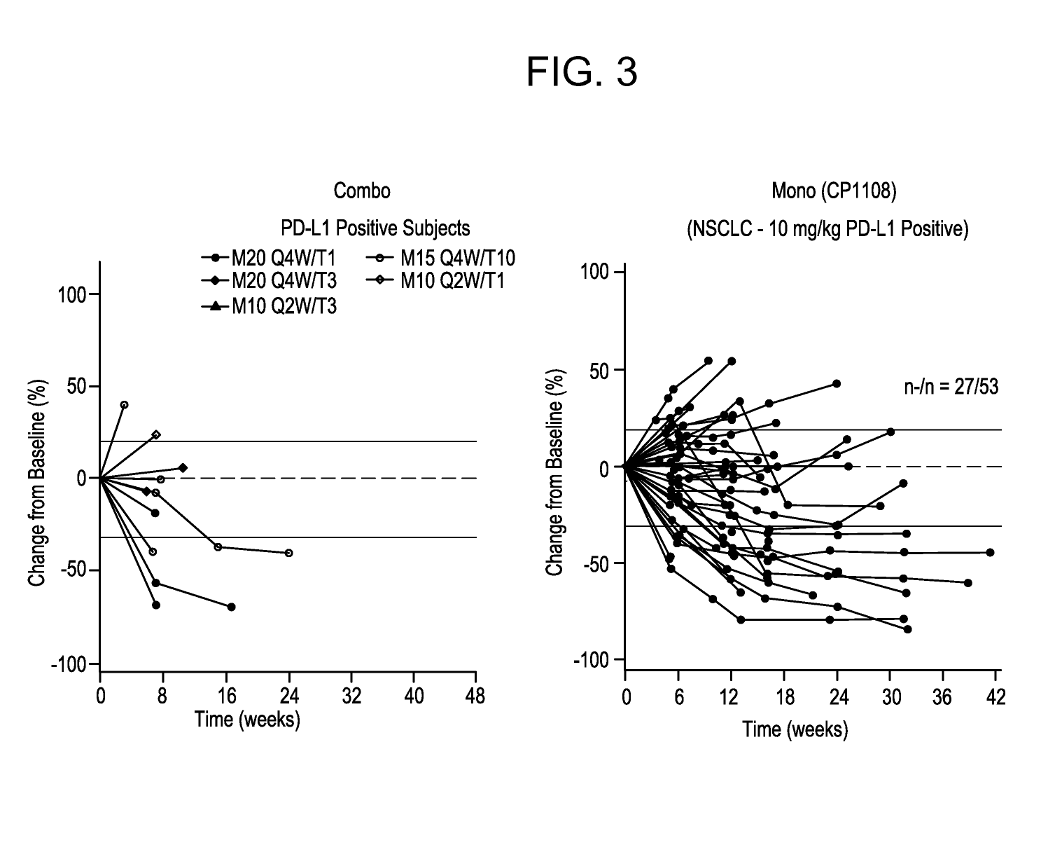

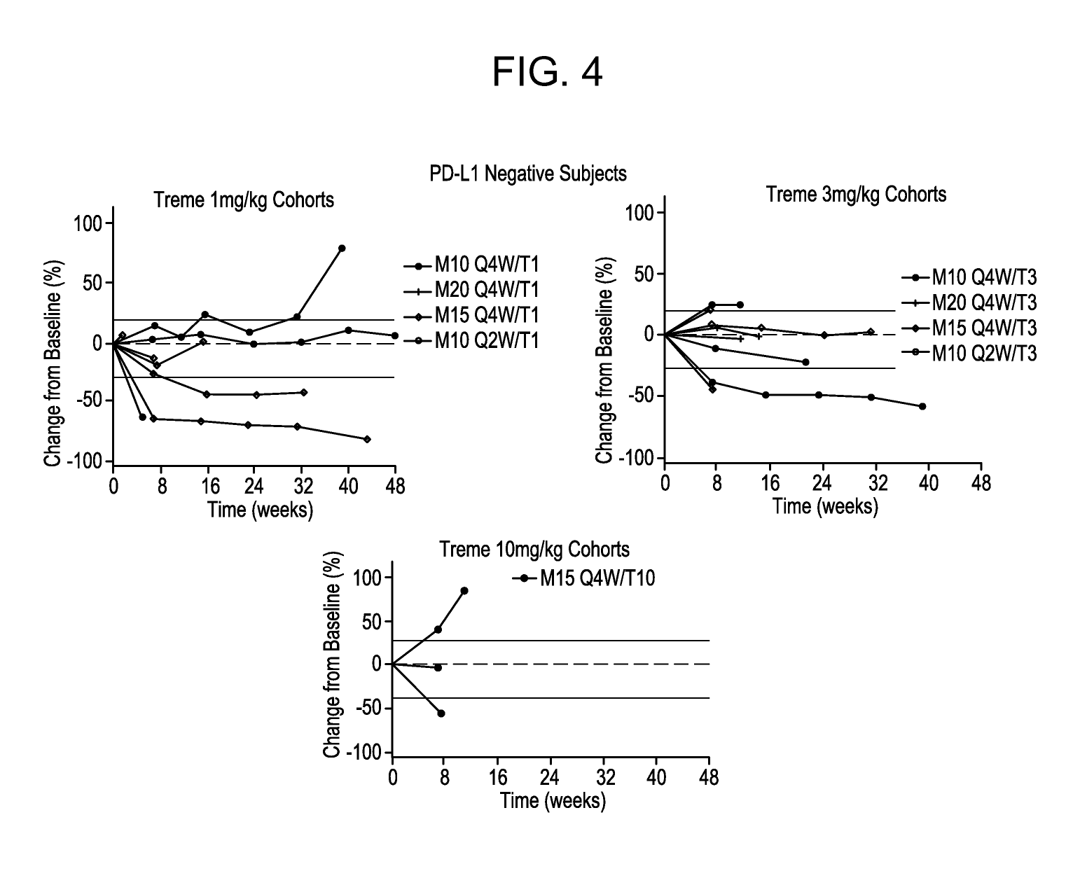

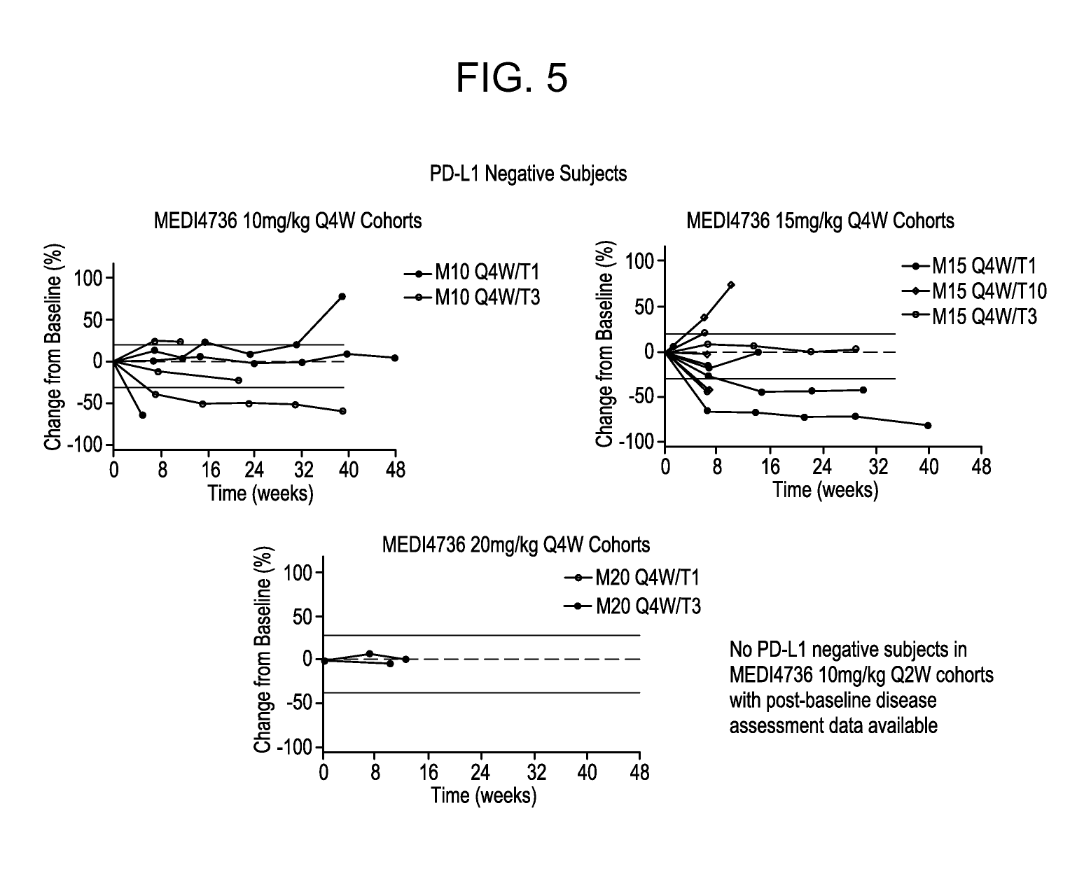

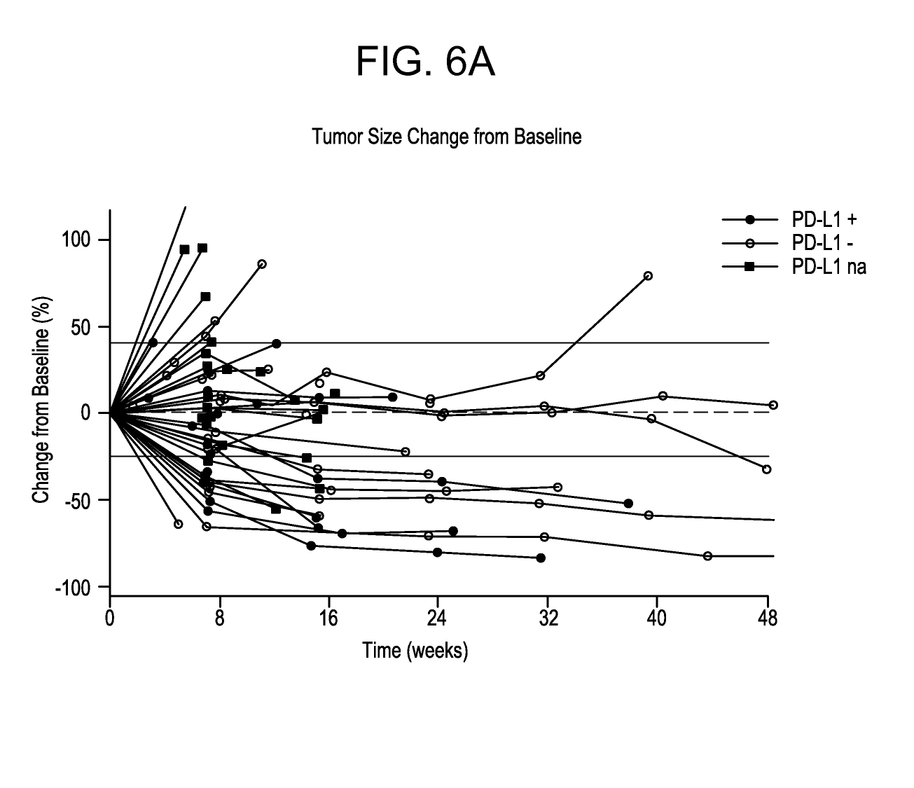

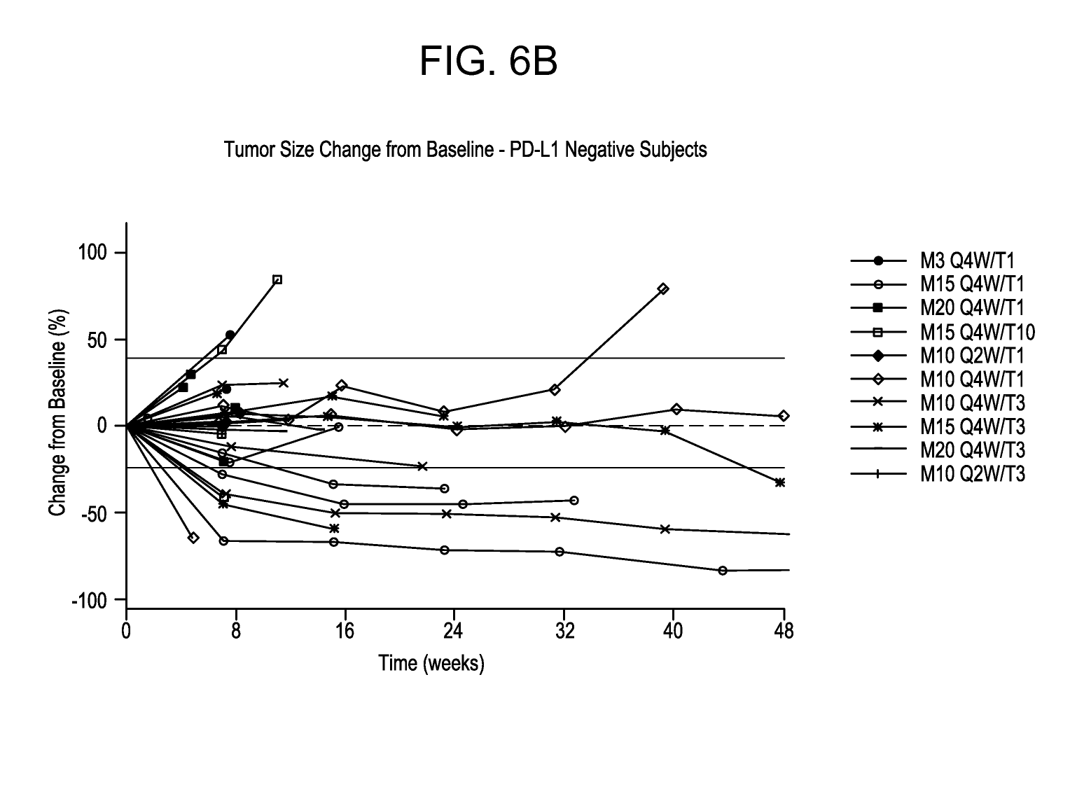

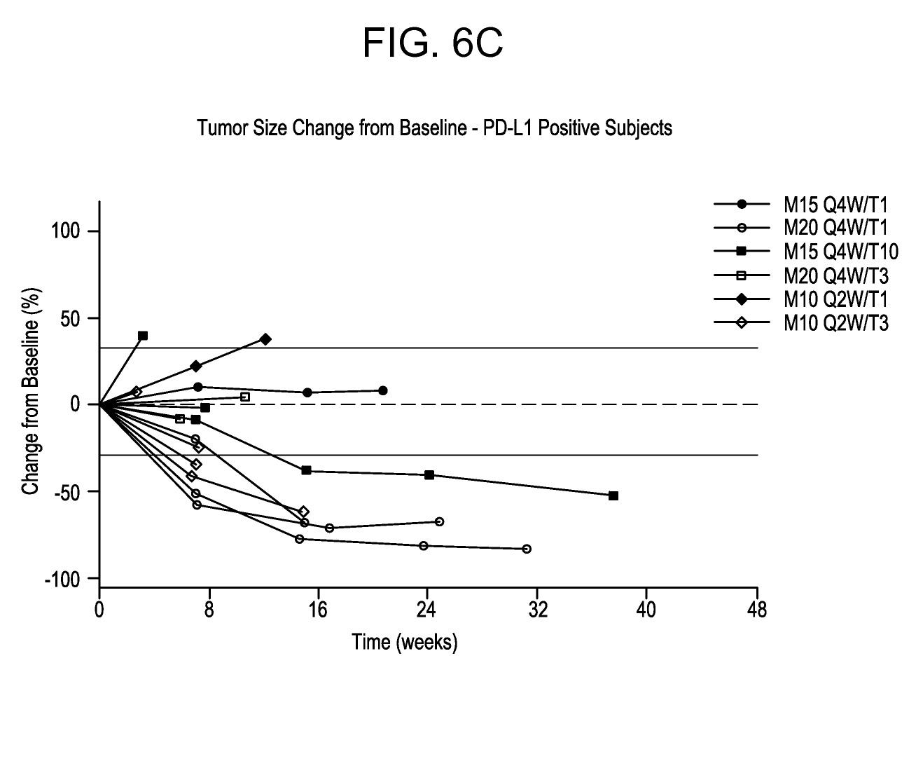

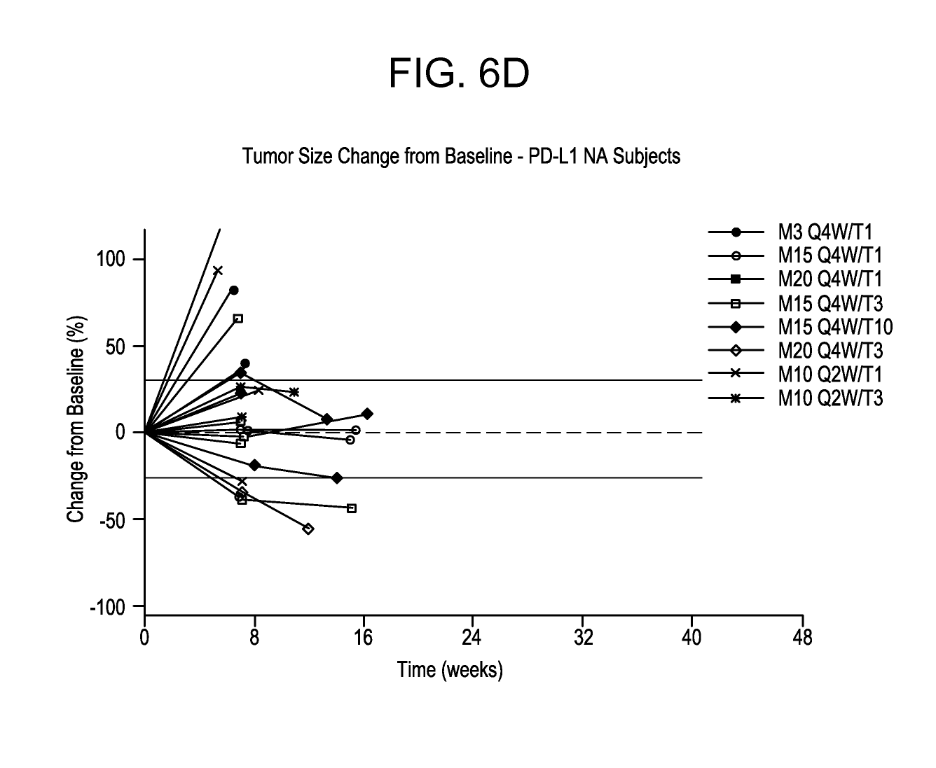

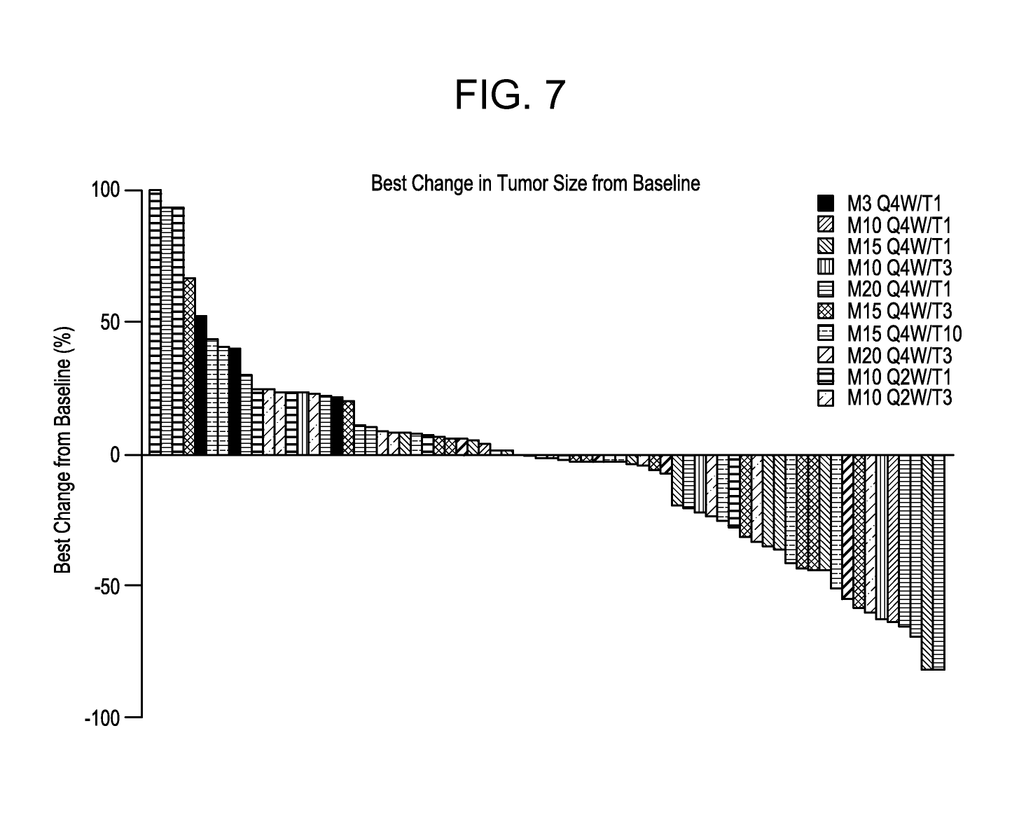

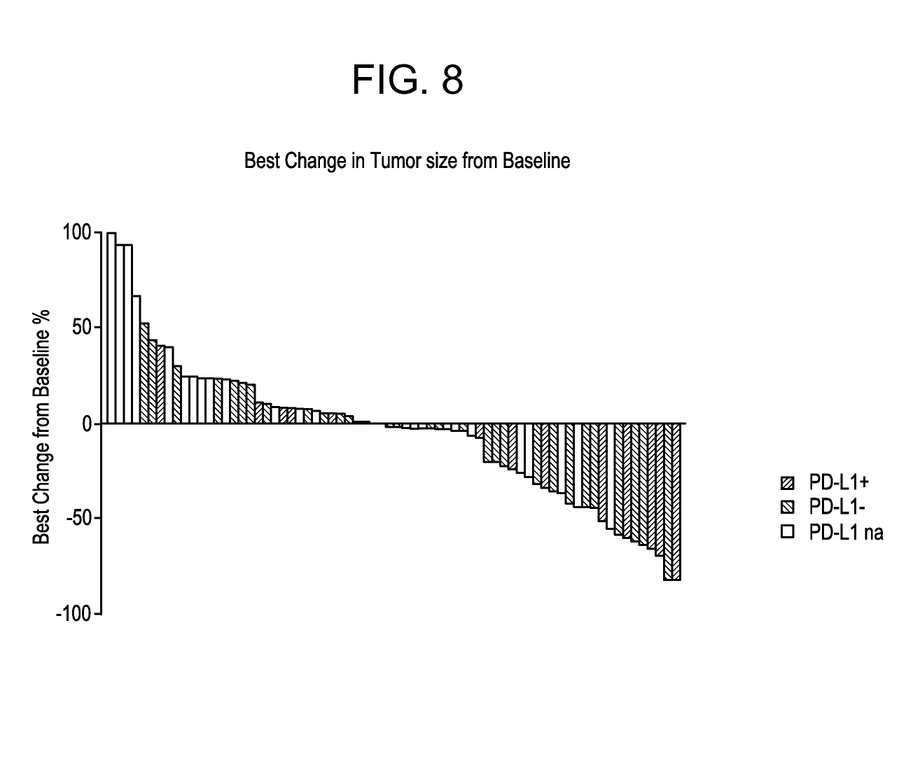

When administered MEDI4736 and tremelimumab, most patients having PD-L1 negative NSCLC responded to combination therapy, and showed decreases in or stabilization of tumor size, compared to MEDI4736 monotherapy (CP1108, 10 mg/kg Q2W) (FIG. 2). Patients having PD-L1 positive NSCLC also responded to the combination of MEDI4736 and tremelimumab compared to MEDI4736 monotherapy, and showed decreases in or stabilization of tumor size (FIG. 3). When the results of the patients having PD-L1 negative NSCLC were grouped by the dose of tremelimumab, 1 mg/kg tremelimumab or 3 mg/kg tremelimumab administered in combination with MEDI4736 at 10 mg/kg Q4W or 15 mg/kg Q4W were effective at controlling or reducing disease (FIG. 4). When the results were grouped by the dose of MEDI4736, the results also showed that tremelimumab at 1 mg/kg to 3 mg/kg administered in combination with MEDI4736 at 10 mg/kg Q4W to 15 mg/kg Q4W was effective at controlling or reducing disease (FIG. 5). Analysis of all NSCLC patients receiving MEDI4736 and tremelimumab showed that PD-L1− and PD-L1+ NSCLC patients responded to treatment (FIGS. 6A-6D, 7, and 8).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

MEDI4736 VL
> PCT/US2010/058007_77 Sequence 77 from PCT/US2010/058007 Organism: Homo
sapiens
SEQ ID NO: 1
EIVLTQSPGTLSLSPGERATLSCRASQRVSSSYLAWYQQKPGQAPRLLIYDASSRATGIPDRFSGSGSGTDFTLTIS
RLEPEDFAVYYCQQYGSLPWTFGQGTKVEIK
MEDI4736 VH
> PCT/US2010/058007_72 Sequence 72 from PCT/US2010/058007 Organism: Homo
sapiens
SEQ ID NO: 2
EVQLVESGGGLVQPGGSLRLSCAASGFTFSRYWMSWVRQAPGKGLEWVANIKQDGSEKYYVDSVKGRFTISRDNAKN
SLYLQMNSLRAEDTAVYYCAREGGWFGELAFDYWGQGTLVTVSS
MEDI4736 VH CDR1
> PCT/US2010/058007_73 Sequence 73 from PCT/US2010/058007 Organism: Homo
sapiens
SEQ ID NO: 3
RYWMS
MEDI4736 VH CDR2
> PCT/US2010/058007_74 Sequence 74 from PCT/US2010/058007 Organism: Homo
sapiens
SEQ ID NO: 4
NIKQDGSEKYYVDSVKG
MEDI4736 VH CDR3
> PCT/US2010/058007_75 Sequence 75 from PCT/US2010/058007 Organism: Homo
sapiens
SEQ ID NO: 5
EGGWFGELAFDY
MEDI4736 VL CDR1
> PCT/US2010/058007_78 Sequence 78 from PCT/US2010/058007 Organism: Homo
sapiens
SEQ ID NO: 6
RASQRVSSSYLA
MEDI4736 VL CDR2
> PCT/US2010/058007_79 Sequence 79 from PCT/US2010/058007 Organism: Homo
sapiens
SEQ ID NO: 7
DASSRAT
MEDI4736 VL CDR3
> PCT/US2010/058007_80 Sequence 80 from PCT/US2010/058007 Organism: Homo
sapiens
SEQ ID NO: 8
QQYGSLPWT
Tremelimumab
> SEQ ID NO: 22 from US 6,682,736
SEQ ID NO: 9
PSSLSASVGDRVTITCRASQSINSYLDWYQQKPGKAPKLLIYAASSLQSGVPSRFSGSGSGTDFTLTISSLQPEDFA
TYYCQQYYSTPFTFGPGTKVEIKRTVAAPSVFIFPPSDEQLKSGTASVVCLLNNFYPREAKV
Tremelimumab VH
> SEQ ID NO: 9 from US 6,682,736
SEQ ID NO: 10
GVVQPGRSLRLSCAASGFTFSSYGMHWVRQAPGKGLEWVAVIWYDGSNKYYADSVKGRFTISRDNSKNTLYLQMNSL
RAEDTAVYYCARDPRGATLYYYYYGMDVWGQGTTVTVSSASTKGPSVFPLAPCSRSTSESTAALGCLVKDYFPEPVT
VSWNSGALTSGVH
Tremelimumab VH CDR1
SEQ ID NO: 11
GFTFSSYGMH
Tremelimumab VH CDR2
SEQ ID NO: 12
VIWYDGSNKYYADSV
Tremelimumab VH CDR3
SEQ ID NO: 13
DPRGATLYYYYYGMDV
Tremelimumab VL CDR1
SEQ ID NO: 14
RASQSINSYLD
Tremelimumab VL CDR2
SEQ ID NO: 15
AASSLQS
Tremelimumab VL CDR3
SEQ ID NO: 16
QQYYSTPFT

1. A method of treatment comprising administering an anti-PD-L1 antibody and an anti-CTLA4 antibody, or antigen binding fragments thereof, to a patient identified as having a lung cancer that is negative for PD-L1.

2. The method of claim 1, wherein the anti-PD-L1 antibody is MEDI4736.

3. The method of claim 1, wherein the anti-CTLA4 antibody is tremelimumab.

4. The method of claim 1, wherein the lung cancer is a non-small cell lung cancer selected from the group consisting of squamous cell carcinoma, adenocarcinoma, large cell carcinoma, adenosquamous carcinoma and sarcomatoid carcinoma.

5. A method of treatment comprising administering MEDI4736 and tremelimumab or antigen binding fragments thereof to a patient identified as having a non-small cell lung cancer that is negative for PD-L1.

6. A method of treatment comprising administering between about 1 mg/kg and 20 mg/kg MEDI4736 and between about 1 mg/kg and 10 mg/kg tremelimumab or antigen binding fragments thereof to a patient identified as having lung cancer that is negative for PD-L1.

7. The method of claim 6, wherein the treatment is administered every 2 weeks, 3 weeks, or 4 weeks.

8. The method of claim 6, wherein the lung cancer is a non-small cell lung cancer selected from the group consisting of squamous cell carcinoma, adenocarcinoma, large cell carcinoma, adenosquamous carcinoma and sarcomatoid carcinoma.

9-10. (canceled)

11. The method of claim 6, wherein about 10 mg/kg MEDI4736 and about 1 mg/kg tremelimumab is administered.

12. The method of claim 6, wherein about 15 mg/kg MEDI4736 and about 1 mg/kg tremelimumab is administered.

13-21. (canceled)

22. The method of claim 6, wherein PD-L1 is detected using immunohistochemistry.

23. The method of claim 22, wherein the immunohistochemistry is carried out on cancer cells that are formalin fixed and paraffin embedded.

24. (canceled)

25. The method of claim 6, wherein the administration of MEDI4736 or an antigen-binding fragment thereof is repeated about every 4 weeks.

26. The method of claim 6, wherein the administration of tremelimumab or an antigen-binding fragment thereof is repeated about every 4 weeks.

27-28. (canceled)

29. The method of claim 6, wherein the administration of MEDI4736 or an antigen-binding fragment thereof is by intravenous infusion.

30. The method of claim 6, wherein the administration of tremelimumab or an antigen-binding fragment thereof is by intravenous infusion.

31-32. (canceled)

33. The method of claim 6, wherein the non-small cell lung cancer expresses reduced or undectable levels of PD-L1.

34. The method of claim 6, wherein the non-small cell lung cancer is negative for PD-L1 when less than 25% of cancer cells show PD-L1 staining.

35-36. (canceled)
